(19) United States
(12) Patent Application Publication
(10) Pub. No.: US 2019/0336206 A1
(43) Pub. Date: Nov. 7, 2019

(54) Electrode Tips For Cardiac Ablation

(71) Applicant: John R. Bullinga, Haverford, PA (US)

(72) Inventor: John R. Bullinga, Haverford, PA (US)

(21) Appl. No.: 16/403,816

(22) Filed: May 6, 2019

Related U.S. Application Data
(60) Provisional application No. 62/666,970, filed on May 4, 2018.

(51) Int. Cl. A61B 18/14

(52) U.S. Cl. CPC A61B 18/1492; A61B 2018/00077

(57) ABSTRACT

Improved electrode tips are provided for cardiac ablation. In accordance with certain preferred embodiments, such tips feature a plurality of conductive portions separated by nonconductive portions to form a plurality of electrodes in the tips. The materials of the tips are biocompatible and capable of sterilization. The same electrode portions may be addressed by a control device both to assess cardiac electrical signals at known cardiac loci and to deliver ablative radiofrequency energy to the same locations. The tips may be provided with perforations to permit irrigation of the catheters when in use.

### CROSS REFERENCE TO RELATED APPLICATIONS

This application claims benefit under 35 U.S.C. § 119(e) of Provisional U.S. Patent Application No. 62/666,970, filed May 4, 2018, the entire contents of which are incorporated herein by reference.

### BACKGROUND

Ablation therapy for heart rhythm disorders (arrhythmias) has become a common therapy for such disorders. This therapy involves identification of an arrhythmia mechanism, determination of critical areas of the arrhythmia circuit, and ablation of critical areas of the arrhythmia circuit. Ablation is typically applied at a catheter tip with the catheter introduced via a femoral vein and the tip manipulated in the heart. Multiple energy sources may be used for ablation including radiofrequency energy, cryothermal, microwaves, and lasers. Radiofrequency ablation is most commonly performed in clinical practice. Safe and effective radiofrequency ablation involves identification of an arrhythmia circuit, localization of critical structures to avoid, application of radiofrequency energy, and monitoring for effective lesion formation. Potential complications of ablation include steam pops, thrombus formation, perforation of cardiac tissue, and damage to collateral structures. The design of the tip of the catheter which applies radiofrequency energy is critical for providing effective localization information and minimizing potential risks of ablation.

Improved electrode tips for cardiac ablation are now provided which achieve one or more of improved safety, effectiveness, efficiency and ease of use.

### SUMMARY

This invention provides new catheter tips which improve the localization of an arrhythmia circuit and greatly reduce the potential risks of ablation when compared to conventional catheters. The new catheter tips are comprised of a plurality of ablation and recording electrodes; each electrode has a dual function of ablation and recording. In accordance with certain preferred embodiments of this invention, ablation can be restricted to the actual sites of recorded signals. Conventional ablation catheters have, for example, a ring electrode proximal to the tip for recording electrical signals from the heart. That ring electrode is not an ablation electrode. Hence, when localizing a circuit using a bipolar electrogram and a conventional catheter, electrical signals from both the ablation tip and the proximal ring electrode contribute to the signal. Localization is less precise in conventional ablation catheters due to the electrical contribution of the proximal ring electrode. Ablation directed at a conventional bipolar electrogram may not result in the desired effect when the ring electrode contributes significantly to the signal. In this situation, ablation applied at the tip may not be directed at the desired tissue producing the signal of interest. This limitation of convention ablation catheter configurations motivates creation of a new catheter dip design with a plurality of electrodes which function for both recording and ablation.

Contact force is distributed with all catheter tips to the targeted ablation tissue. The present, improved catheter tips are designed with a generally spherical shape to minimize focal contact forces which could lead to tissue disruption. By having a substantially spherical shape, contact force, regardless of catheter orientation, will be distributed about the targeted tissue.

### DESCRIPTION OF THE DRAWINGS

FIG. 1 illustrates a catheter tip comprised of two conductive and two nonconductive regions. The conductive tip is perforated to allow cooling irrigant. A nonconductive region separates the two thermally conductive regions.

FIG. 2 illustrates a catheter tip comprised of two thermally conductive regions separated by a nonconductive region. This allows for bipolar electrical signal acquisition between the thermally conductive regions. This also allows for radiofrequency energy delivery from each of the conductive regions such that ablation is delivered at the same site as electrical recording. The catheter tip maintains a substantially spherical shape, minimizing inhomogeneity of catheter forces applied to cardiac tissue. This design is highly effective as an ablation tip.

FIG. 3 illustrates a catheter tip comprised of three thermally conductive regions separated by nonconductive material. This allows for multiple bipolar electrical signal acquisition between each of the thermally conductive regions.

FIG. 4. Illustrates a catheter tip comprised of four electrically and thermally conductive regions separated by a nonconductive material. In this design, the electrically and thermally conductive regions are placed equally at 90° along the axis of the catheter tip.

FIG. 5. Illustrates a catheter tip comprised of four electrically and thermally conductive regions separated by a nonconductive material. In this design, the electrically and thermally conductive regions are placed equally at 120° along the axis of the catheter tip with the fourth electrode placed at the distal portion of the catheter tip.

FIG. 6. Illustrates a catheter tip comprised of five electrically and thermally conductive regions separated by nonconductive material. In this design, the electrically and thermally conductive regions are placed equally at 90° along the axis of the catheter tip with the fifth electrode placed at the distal portion of the catheter tip.

FIG. 7. Illustrates a split ring ablation and recording element. This ring is designed to include two electrically and thermally conductive regions separated by a non-conductive region. This element is intended to allow for ablation along the shaft of an ablation catheter while simultaneously allowing for improved recording of electrograms.

FIG. 8. Illustrates the same configuration of two electrically and thermally conductive regions. This is the same overall configuration as FIG. 2. Rather than being an irrigated distal electrode, the tip is solid.

FIG. 9. Illustrates a catheter tip comprised of two conductive regions separated by non-conductive regions. This format does not exemplify a substantially spherical geometry at the tip, but, rather, one which is substantially in the shape of a canister. The canister shape in this embodiment has carefully rounded edges so as to reduce the risk of injury to tissue to which it may be applied.

### DETAILED DESCRIPTION

The present invention may be further understood with reference to the following description and the appended drawings, wherein like elements are provided with the same reference numerals. The present invention includes an ablation catheter tip that has multiple elements which serve the functions of recording electrical signals of the heart and delivering electrical energy to the heart for ablation of cardiac tissue. The presence of multiple recording and ablation elements at the ablation tip allows for restricting the recording of cardiac signals to the local area of tissue undergoing ablation and allows for an improved assessment of ablation of tissue.

As will be apparent, catheter tips in accordance with some embodiments of the invention comprise both electrically conductive and nonconductive materials. As the catheters of the invention are invasive devices, all of the materials comprising them must be sterilizable and compatible with the intracorporeal uses intended for them. In practice, a wide variety of materials may be employed as conductive materials for use in the invention. Conveniently, metallic materials are both suitable and appropriate. It will be appreciated that metallic elements, alloys and blends, which are biocompatible and temperature stable to sterilizing conditions may be employed. Steels, brasses, titanium structures and alloys, magnesium and aluminum materials, alloys and blends and, indeed, a large variety of metallic structures may serve this function.

Particular examples of conductive metals useful in the invention include stainless steel, aluminum and titanium. Alloys include those which comprising iron, nickel, cobalt, chromium, zinc, tin, titanium, molybdenum, silver, platinum, gold, manganese and palladium. It may also be useful to formulate conducting regions or portions of the tips from conductive ceramic, rubber or plastic. In this regard, ceramic, containing one or more metallic filaments, fillers or other materials which render the hard mass of ceramic effectively conducting, may find use herein. Certain polymers and rubbers, such as polysiloxanes and high molecular weight silicones are temperature stable and biocompatible. They may also be rendered conductive through inclusion of conductive filaments, fibers or fillers therein such as carbon, especially nanostructured carbon, metal, graphite or other conductive materials. Other conducting materials which may be suitable may be found in U.S. Pat. No. 8,211,102, incorporated herein by reference.

The improved catheter tips of the invention also include regions or portions which are relatively non-conducting. In this context, plastics, rubbers and ceramics are generally preferred. As with the other materials of the catheter tips, biocompatibility and temperature stability are desired. Exemplary materials for formulation of the composite electrode tips hereof include biocompatible and thermally stable epoxys, polyurethanes, and mixed urethane/epoxys. Silicone and polysiloxanes materials may be used as may polyfluorinated species. Nonconducting ceramics can be useful, especially when co-sintered with areas of conducting ceramic.

Mechanical perforation of the heart with a catheter tip may result from excessive contact force applied to the catheter tip against a portion of the heart. Perforation risk may be a function of the total tip force per total tissue contact area where higher forces per area result in a higher risk of mechanical perforation. Also, the shape of the catheter tip may influence the propensity for mechanical perforation. A catheter shape which produces a higher local tip force per local tissue contact area may disrupt a tissue plane and produce a higher risk of perforation than a catheter shape which minimizes local tip force per local tissue contact area.

The ideal catheter tip shape may be substantially spheroid so that the curvature of the catheter tip is generally constant along all directions of the surface and at all points of the surface. A sphere has one curvature valued which is a function of the radius of the sphere. The tip contact shape will be the same and independent of the catheter orientation relative to the cardiac tissue. This minimizes the risk catheter tip shape causing an increase in local tip force per local tissue contact area and resulting in a higher perforation risk. Ovoid characteristics may be employed to some degree consistent with the foregoing considerations.

Cooling of a catheter tip during radiofrequency ablation is accomplished by convective heat loss. One method to increase convective heat loss is to increase the size of the ablation electrode resulting in a larger surface to disperse heat. Convective heat loss may also be increased by irrigating the catheter tip with a fluid. External irrigation refers to cooling a catheter tip using a fluid pumped through from the proximal portion of the catheter and released into the body of the patient at the distal ablation portion of the catheter. Internal irrigation refers to cooling a catheter tip using a fluid pumped through from the proximal portion of the catheter, circulating through the distal ablation portion of the catheter, and returning to the proximal portion of the catheter for removal. Either mechanism may be employed in conjunction with the present invention.

The size of the electrode influences the electrogram recorded from the electrode. A smaller electrode results in the ability to record more localized cardiac electrical signals whereas a larger electrode results in an averaging over an area of cardiac electrical signals and a less localized cardiac electrical signal. In effect, the shape and size of the recording electrode act as a filter of cardiac electrical signals. Standard ablation catheters conventionally have a 7-8 French diameter and are 3.5-10 millimeters in length. Recording from large electrodes results and filtering of localized cardiac electrical signals such that high-frequency fractionated electrograms are filtered to reduced amplitudes. However these high-frequency fractionated electrograms are also signals of interest when performing catheter ablation. Thus conventional catheter ablation tips which have relatively large tips are not ideal for recording cardiac signals to direct ablation.

In one preferred form of this invention, the overall size of the catheter tip ablation surface is similar to standard conventional ablation tips. However, the tip itself is divided into two or more conductive regions separated by nonconductive material. Each of the conductive regions may be seen to be electrodes, which may be used for recording cardiac electrical activity and for delivering energy such as radiofrequency energy. While the overall size of the catheter tip is similar to conventional catheters, the size of the recording electrode or electrodes is smaller. The catheter tip, thus, will have the ability to record more localized and higher frequency fractionated electrograms. These are the electrograms commonly of interest when performing ablation. Thus the catheter tips now provided have superior recording characteristics compared to other conventional catheters.

Prior work and interventions have included using microelectrodes which are electrically isolated from the electrode delivering radiofrequency energy. In those works, the microelectrodes did not have the dual purpose of recording electrical activity and delivering radiofrequency energy. Thus, this invention is distinctly different from these prior devices.

Conventional ablation catheters have a distal ablation electrode and a ring electrode proximal to the ablation electrode for recording. By having the ring electrode relatively close to the ablation electrode, the resulting bipolar signal is relatively localized to the site of ablation. When analyzing catheter signals, the distal bipolar signal is commonly used to find the best site for ablation. However, both the distal ablation electrode and the non-ablation proximal ring electrode both contribute to the bipolar signal. If the actual site of optimal ablation is adjacent to the non-ablation proximal ring electrode and not the distal ablation electrode and ablation is directed by the bipolar electrogram, then the operator may direct ablation at a site which is not ideal for treatment of an arrhythmia. In effect, the optimal recording site from a conventional ablation catheter may not be the same as the optimal ablation site. This may increase risks to the patient due to more ablation being required to treat the arrhythmia since there is less accuracy if identification of the optimal ablation site with standard bipolar electrogram analysis. The ideal configuration of an ablation catheter is to have both electrodes contributing to a bipolar electrogram being ablation electrodes. Thus, the optimal recording site may or may not be the optimal ablation site. The present catheter tip designs address this issue by having more than one ablation and recording electrode forming the catheter tip.

One of the measures of successful ablation is the voltage loss of the bipolar electrogram during ablation. The loss of bipolar voltage reflects a loss of local excitability of the cardiac tissue as a result of ablation. As discussed above, the conventional catheter design has a bipolar recording from the distal ablation electrode and the non-ablation proximal ring electrode. Residual voltage recorded from the non-ablation proximal ring electrode may result in an operator believing that additional ablation needs to be performed although ablation may have been successfully performed at the distal tip. The ideal configuration of an ablation catheter is to have both electrodes contributing to a bipolar electrogram being ablation electrodes. Thus, residual voltage from the bipolar recording of the ablation electrodes reflects local excitable tissue and a less complete ablation and loss of voltage from the ablation electrodes reflecting successful ablation. This new catheter tip designs address this by having more than one ablation and recording electrode within the catheter tip.

A second measure of ablation success is failure to capture local tissue with pacing. Pacing may be performed in a unipolar or bipolar configuration. Unipolar pacing results in energy delivery to the selected electrode, however may result in saturation of multiple recording signals making analysis of electrograms more difficult. Bipolar pacing results in energy delivery to a pair of electrodes and both electrodes may result in local tissue excitation. Since the measure of success of ablation is whether or not pacing results in tissue excitation either the distal ablation electrode and the non-ablation proximal ring electrode may result in tissue excitation, a false impression that additional ablation is required may occur due to tissue excitation from the non-ablation proximal ring electrode. The ideal configuration of an ablation catheter is to have both electrodes contributing to a bipolar electrogram being ablation electrodes. Thus, pacing induced tissue excitation from bipolar pacing is a result of locally viable tissue adjacent to an ablation electrode. The new catheter tip designs address this issue by allowing for bipolar pacing from ablation electrodes within the ablation tip.

It is desirable to have the ability to record a more localized signal so that ablation of cardiac tissue may be directed to the best possible location. Recording of more localized signals requires a small electrode; however a smaller electrode results in more rapid heating of the electrode which limits power delivery. Improved convective heat loss using external or internal irrigation may allow for a smaller electrode and improved signals. However, there remain issues of bipolar recording and pacing from a non-ablation electrode and a small electrode size producing a higher force per tissue area and irregular tip electrode shapes producing higher localized force per local tissue area.

The improved ablation catheters of this invention preferably have more than one ablation and recording electrode on a catheter tip, which catheter tip is substantially spherical. Additionally, irrigation of the catheter tip, while optional, allows for the total electrode size to be relatively small while allowing for adequate power delivery to result in ablation lesions. Since more than one ablation and recording electrode is on the catheter tip, the tip's diameter may be increased in size to accommodate the electrodes. The larger diameter of the catheter tip reduces the risk of mechanical perforation of the heart during a procedure. Since the size of the electrodes is small, signal quality is maintained or improved when compared to conventional catheters.

It will be understood that preferred catheter tips featured in this invention are “substantially spherical”. This does not require that they be perfectly spherical in the geometric sense. Indeed, a point of attachment of the tip to the rest of the electrode is necessary and such defects perfect sphericality. Moreover, non-spheroidal character may be included to a degree. Thus, some hybrid shapes including ovoid, elliptical or other geometrical elements may form a blended geometry for the tips. In all cases, it will be appreciated that relatively smooth surfaces having relatively large radii will be presented to the sites of sensing and ablation.

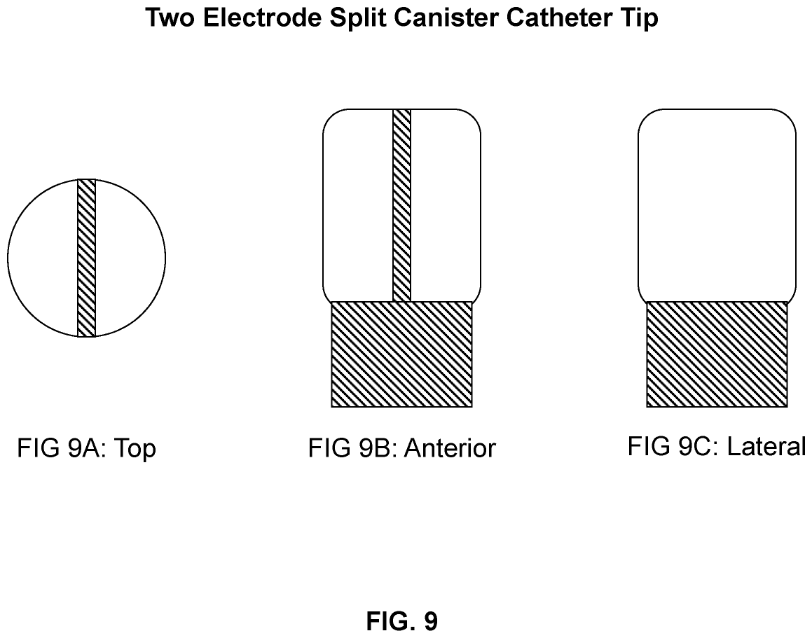

It may be desirable for some applications to include the design element of multiple separated electrodes within a tip of a non-spherical catheter tip. FIG. 9 is an illustration of a canister shaped tip with rounded edges. Deployment of a tip through a catheter shaft may limit the size of the deployable catheter tip and make a non-spherical tip desirable. The design feature of large separate conductive regions to serve as electrodes for recording and energy delivery is desirable on any shape of a catheter tip. Significant rounding of the canister edges is desirable for the reasons discussed for the spherical tip design.

It has been found to be particularly useful to employ composite, irrigated tips on electrons, which are comprised of two materials. An insulator material is used to separate electrodes. A conductor material is used as electrodes and for ablation. The simplest form is to have two electrodes each with a thermister. The tip may have a conventional shape or have the preferred spherical end shape to reduce contact forces. The tip is split by insulating material along the vertical axis. The two electrodes can be used for a bipolar recording and all portions of the electrodes may be used for ablation. Hence, any electrogram associated with that bipole reflects electrically active tissue. Both materials are preferably, substantially uniformly perforated for irrigation. In one preferred design, the tip is in the shape of a ball, which minimizes contact forces and reduces the risk of perforation.

Additional electrodes may be added if desired. Conductor materials will be arranged in a geometric pattern around the tip. It will also help maximize tissue contact. Contact sensing using a variety of methods may be used to determine which tip electrodes should receive ablation power.

The conductive electrode portions of the catheter tips of the invention are formed so that they may be placed into electrical communication with a control device. The control device is preferably capable of receiving sensing signals from the conductive portions and for driving ablative radiofrequency energy to those same conductive portions. The control device is conventionally supplied with power from a power supply, control, preferably via a digital computational function and suitable connectors. Preferably, each conductive electrode is separately connected to the control device for independent sensing and ablation.

What is claimed:

1. A catheter tip comprising a plurality of biocompatible, conductive elements separated by biocompatible, nonconductive material, the conductive elements being independently capable of recording electrical signals from cardiac tissue when in the vicinity of said tissue and of delivering radiofrequency energy to said cardiac tissue.

2. The catheter tip of claim 1 having a substantially spherical shape.

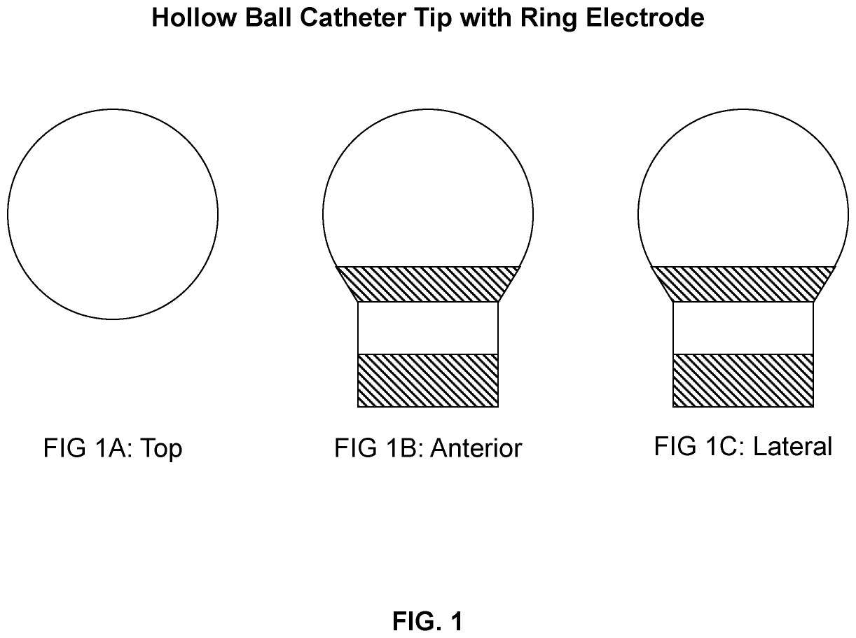

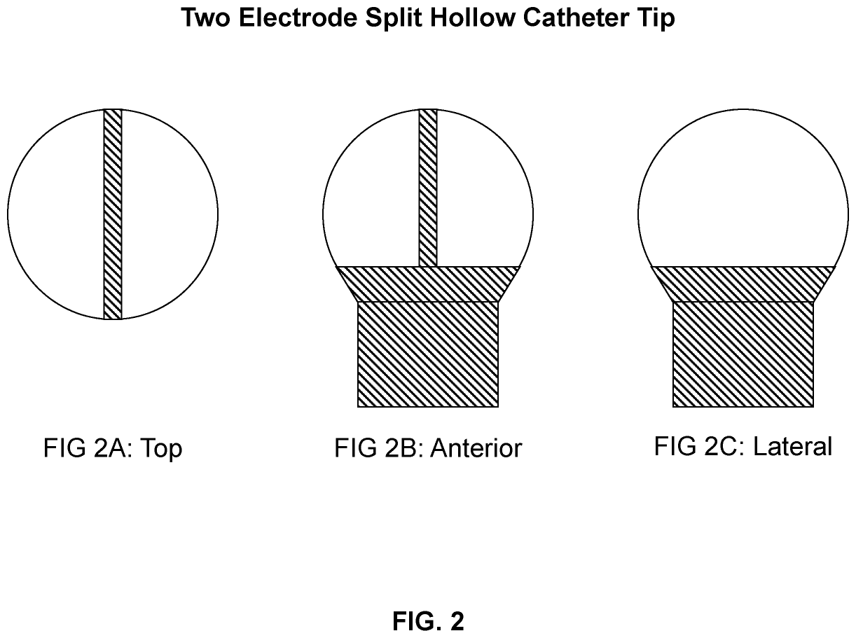

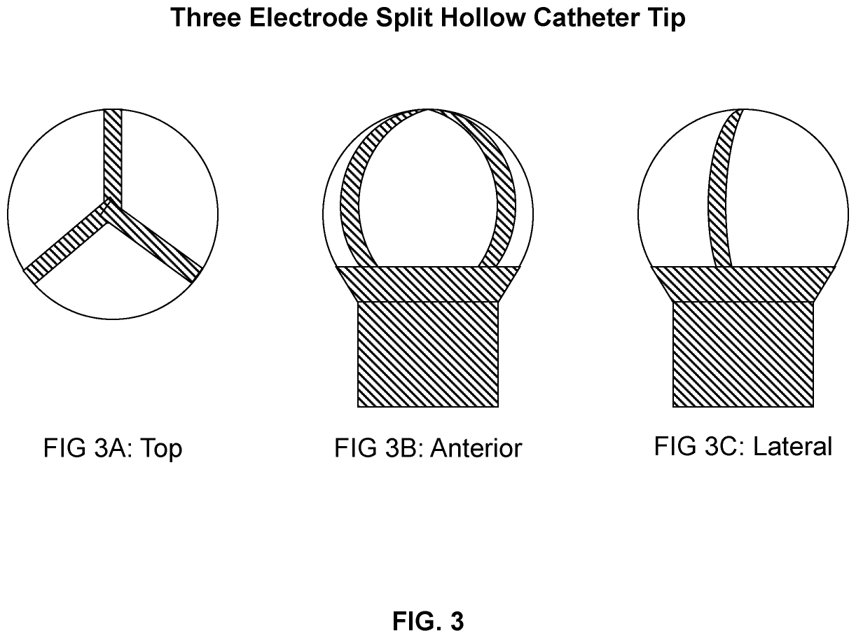

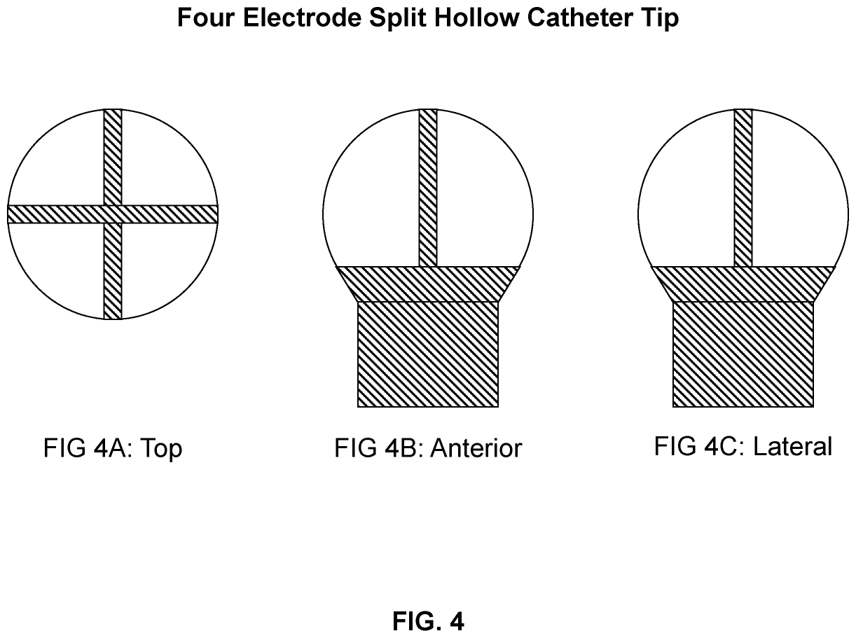

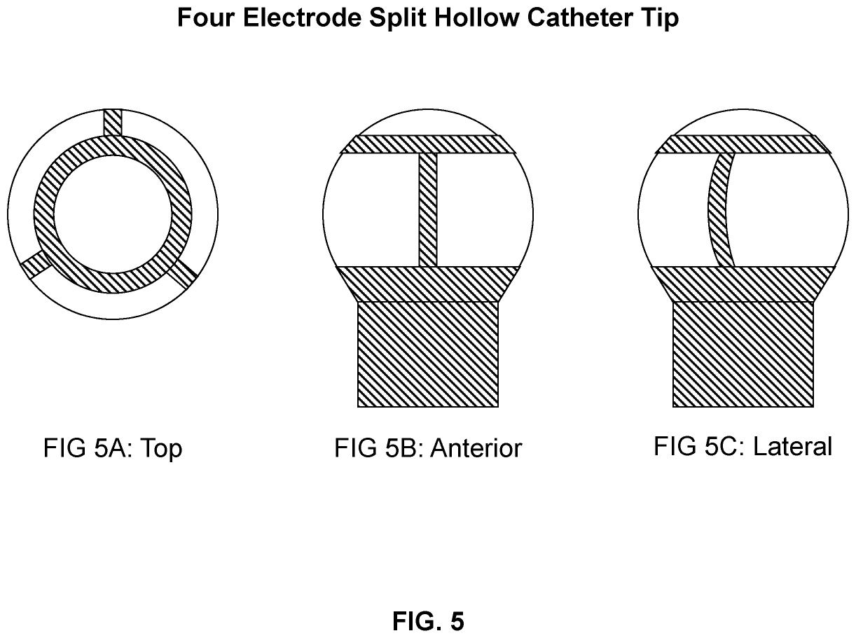

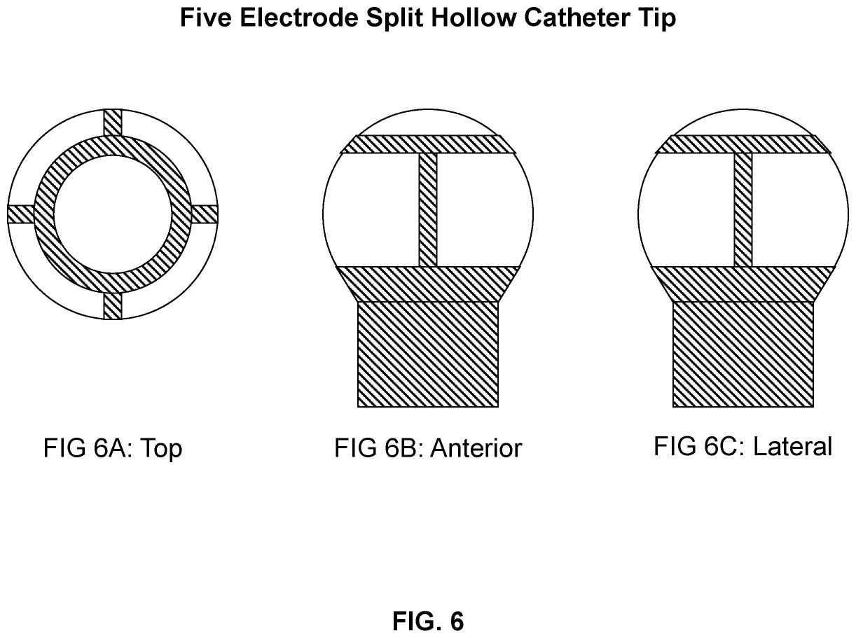

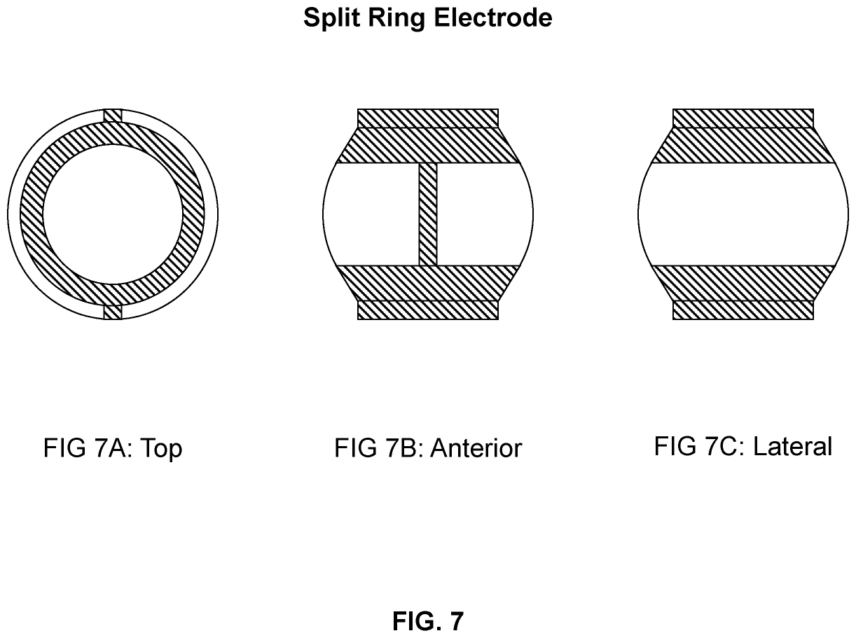

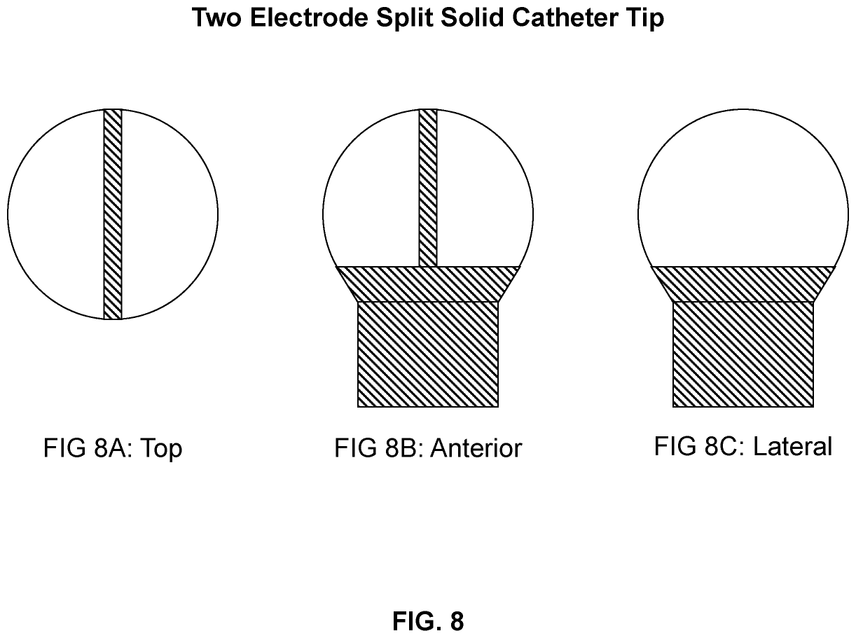

3. The catheter tip of claim 1 wherein the tip is formed and disposed in accordance with any of FIGS. 1-8 hereof.

4. The catheter tip of claim 1 where the tip is perforated.

5. The catheter tip of claim 5 adapted for the passage of irrigation fluid through the perforations.

6. The catheter tip of claim 1 further comprising a temperature sensor.

7. A catheter tip comprising a plurality of biocompatible, conductive elements separated by biocompatible, nonconductive material, the conductive elements being independently capable of recording electrical signals from cardiac tissue when in the vicinity of said tissue and of delivering radiofrequency energy to said cardiac tissue, the catheter tip having a substantially canisterial shape.

8. The catheter tip of claim 1 wherein the tip is formed and disposed in accordance with FIGS. 9 hereof.
